(19) United States
(12) Patent Application Publication
(10) Pub. No.: US 2020/0060592 A1
(43) Pub. Date: Feb. 27, 2020

(54) SENSORS AND METHODS FOR MEASURING pH

(71) Applicant: Abbott Diabetes Care Inc., Alameda, CA (US)

(72) Inventors: Benjamin J. Feldman, Berkeley, CA (US); Suyue Qian, Fremont, CA (US)

(73) Assignee: Abbott Diabetes Care Inc., Alameda, CA (US)

(21) Appl. No.: 16/546,731

(22) Filed: Aug. 21, 2019

Related U.S. Application Data
(60) Provisional application No. 62/721,701, filed on Aug. 23, 2018.

(51) Int. Cl. A61B 5/1473; A61B 5/145

(52) U.S. Cl. CPC A61B 5/14735; A61B 5/14539

(57) ABSTRACT

It can be particularly difficult to measure pH in vivo using current electrochemical sensors due to sensor drift and fouling of the sensor surface. Sensors suitable for measuring pH, particularly in vivo, may comprise: a sensor tail comprising a first working electrode, a second working electrode, and at least one other electrode; a first active portion located upon the first working electrode, the first active portion comprising a substance having pH-dependent oxidation-reduction chemistry; and a second active portion located upon the second working electrode, the second active portion comprising a substance having oxidation-reduction chemistry that is substantially invariant with pH. A difference between a first signal from the first active portion and a second signal from the second active portion may be correlated to a pH value for a fluid.

### BACKGROUND

The detection of various analytes within an individual can sometimes be vital for monitoring the condition of their health and well-being. Deviation from normal analyte levels can often be indicative of an underlying physiological condition, such as a metabolic condition or illness.

Analyte monitoring in an individual may take place periodically or continuously over a period of time. Periodic analyte monitoring may take place by withdrawing a sample of bodily fluid, such as blood, at set time intervals and analyzing ex vivo. Continuous analyte monitoring may be conducted using one or more sensors that remain at least partially implanted within a tissue of an individual, such as dermally, subcutaneously or intravenously, so that analyses may be conducted in vivo. Implanted sensors may collect analyte data at any dictated rate, depending on an individual's particular health needs and/or previously measured analyte levels.

Periodic ex vivo analyte monitoring can be sufficient to determine the physiological condition of many individuals. However, ex vivo analyte monitoring may be inconvenient or painful for some persons. Moreover, there is no way to recover lost data if an analyte measurement is not obtained at an appropriate time.

Continuous analyte monitoring with an in vivo implanted sensor may be a more desirable approach for individuals having severe analyte dysregulation and/or rapidly fluctuating analyte levels, although it can also be beneficial for other individuals as well. While continuous analyte monitoring with an implanted sensor can be advantageous, there are challenges associated with these types of measurements. Intravenous analyte sensors have the advantage of providing analyte concentrations directly from blood, but they are invasive and can sometimes be painful for an individual to wear, particularly over an extended period. Subcutaneous, interstitial or dermal analyte sensors can often be less painful for an individual to wear and can provide sufficient measurement accuracy in many cases.

Any analyte may be suitable for analysis in vivo provided that a suitable chemistry can be identified for sensing the analyte. Indeed, in vivo amperometric sensors configured for assaying glucose have been developed and refined over recent years. Other analytes commonly subject to physiological dysregulation that may similarly be desirable to monitor include, but are not limited to, lactate, oxygen, pH, A1c, ketones, drug levels, and the like.

In vivo pH levels typically remain within a fairly narrow range for normal biological functioning to take place. Normal blood pH, for example, is about 7.4, and blood pH values of less than 6.9 or greater than 7.6 may be life threatening. Consequences of biological pH dysregulation include, but are not limited to, in vivo precipitation of one or more components within a biological fluid, enzyme hypoactivity or hyperactivity, altered biomembrane permeability, propensity toward some types of cancer, and other undesirable conditions. Examples of some conditions that can lead to an offset in pH measurements include respiratory acidosis, respiratory alkalosis, metabolic acidosis, and metabolic alkalosis. Regarding acidosis, respiratory acidosis may be caused by, among other things, chest deformity or injury, chronic lung and airway disease, overuse of sedatives, or obesity. Metabolic acidosis may be caused by, among other things, prolonged exercise, lack of oxygen, certain medications, including salicylates, low blood sugar, alcohol, seizures, liver failure, some cancers, kidney disease, severe dehydration, and poisoning (e.g., methanol poisoning). Regarding alkalosis, respiratory alkalosis may be caused by, among other things, lack of oxygen, fever, lung disease, liver disease, and salicylate poisoning. Metabolic alkalosis is rare but may be caused by, among other things, severe dehydration, cystic fibrosis, and overuse of alkalotic agents, such as antacids.

In addition to the health consequences directly attributable to dysregulated in vivo pH values, sensing chemistries associated with certain analytes also may be influenced by the local pH environment and/or change in the local pH environment. Urea detection, for example, may be based upon the pH changes that occur when interacting a biological fluid with urease to produce ammonia as a product. The ammonia changes the local pH environment, and measurement of the pH may allow the urea concentration to be assayed. Without having an accurate way to measure pH, however, it can be difficult to obtain reliable concentration measurements for urea and similar analytes, even when an in vivo pH level is not directly affecting the analyte concentration itself.

Measurement of pH levels in an individual is conventionally performed by withdrawing biological fluid samples at set time intervals and analyzing ex vivo. While this approach may be acceptable in certain instances, in the case of rapidly changing pH levels, it may be difficult to measure pH levels with sufficient rapidity to determine that pH dysregulation has occurred. Moreover, because there is frequently a time lag associated with obtaining ex vivo pH measurements, significant health consequences may have taken place by the time it becomes apparent that pH dysregulation has occurred.

While it might be desirable to measure pH levels in vivo, particularly with a single implanted sensor over extended measurement times, the nature of conventional pH measurements makes this task difficult. First, conventional pH measurements are typically made using glass electrodes or ion sensing field effect transistors (ISFETs). Both types of devices are extremely sensitive to surface fouling, which may affect the measured surface potential and therefore the measured pH. Second, reference electrodes used in conjunction with measuring pH are subject to drift, especially in vivo, which results in a further source of measurement error. Reference electrodes for in vivo measurement of analytes, such as glucose, for example, may be operated amperometrically at a plateau potential and are much less subject to drift effects, in contrast. As such, conventional approaches are not especially well suited for measuring in vivo pH values, particularly over extended measurement times.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIGS. 1A and 1B show illustrative configurations of a pH sensor having two working electrodes and a counter/reference electrode, according to the various embodiments described herein.

FIG. 2 shows an illustrative configuration of a pH sensor having two working electrodes, a reference electrode and a counter electrode, according to the various embodiments described herein.

FIG. 3 shows a diagram of an illustrative sensing system adapted for on-body wear and capable of measuring pH based upon receipt of signals from first and second working electrodes, according to the various embodiments described herein.

FIG. 4 shows a plot of aggregate cyclic voltammograms at various pH values for a working electrode containing polymer-bound toluidine blue.

FIG. 5 shows a corresponding plot of aggregate cyclic voltammograms at various pH values for a working electrode containing a polymer-bound osmium complex.

FIG. 6 shows a calibration curve corresponding to the pH versus voltage difference data shown in Table 1.

### DETAILED DESCRIPTION

The present disclosure generally describes sensors and methods for measuring pH and, more specifically, sensors and methods especially suited for measuring pH values in vivo.

As discussed above, in vivo electrochemical measurement of pH values may be complicated due to drift and surface fouling of one or more electrodes or similar pH measurement devices. Although these issues may be particularly problematic in vivo, it is to be appreciated that they sometimes may also be encountered when measuring pH values ex vivo or otherwise in a laboratory setting. Without the ready ability to measure pH values accurately in vivo, it can be difficult to assess an individual's physiological condition in real-time or near real-time. Moreover, the sensing chemistry associated with measuring the concentration of certain analytes may be pH-dependent, and inaccurate analyte concentration measurements may arise from an inability to measure pH with sufficient accuracy.

In the present disclosure, pH sensors are described that overcome the above-referenced challenges and may provide additional benefits as well. In particular, the pH sensors of the present disclosure are largely unaffected by the effects of surface fouling and drift. The pH sensors of the present disclosure employ two different working electrodes, in which an active portion of each working electrode exhibits different electrochemical performance. As used herein, the term “active portion” refers to a layer or spot(s) located upon a portion of a working electrode where a desired electrochemical reaction takes place. Namely, in the pH sensors of the present disclosure, the active portion located upon the first working electrode comprises a substance having pH-dependent oxidation-reduction chemistry, and the active portion located upon the second working electrode comprises a substance having oxidation-reduction chemistry that is substantially invariant with pH. According to some embodiments, the substance located in the active portion of the first working electrode may comprise a substance that changes its protonation state in the course of an oxidation-reduction reaction, although this is not a requirement. The potential at which the oxidation state changes may be determined during a voltammetric sweep of the first working electrode. According to some or other various embodiments, the substance located in the active portion of the second working electrode need not necessarily have a completely invariant potential measured during a voltammetric sweep over a given pH range of interest. It is to be appreciated that the change in measured voltage may be less than a predetermined suitable value over a given pH range, such as less than about 100 mV variability or less than about 50 mV variability, or less than about 10 mV variability over a given pH range of interest. The variance may be determined by subtracting the minimum voltage from the maximum voltage observed over the voltage sweep between the extremes of the useful pH range. The acceptable amount of variance may be determined based upon how accurate the pH measurement is required to be. According to some embodiments, the substance located in the active portion of the second working electrode may maintain (not change) its protonation state in the course of undergoing an oxidation-reduction reaction, but this again is not a requirement.

Signals may be received from the first working electrode and the second working electrode in order to calculate a pH value for a fluid in contact with the pH sensor. In particular, an observed pH value for the fluid may be calculated based upon a difference between the first signal and the second signal. The signal difference may be correlated to a pH value (e.g., with a suitable processor or manually) by consulting a lookup table, calibration curve, or the like.

Although the pH sensors disclosed herein may comprise a reference electrode and a counter electrode or a counter/reference electrode, it is not necessary to receive, reference, utilize, or otherwise process a signal from the reference electrode or the counter/reference electrode to determine a pH value. Namely, since the first working electrode and the second working electrode are individually referenced against the reference electrode or the counter/reference electrode, the correction applied to the first and second signals cancels out when determining the signal difference. Stated differently, the first working electrode and the second working electrode are internally referenced against each other. Accordingly, the pH sensors described herein overcome the drift issue associated with conventional pH sensors by eliminating the need for signal correction using a reference electrode or a counter/reference electrode. Moreover, given that a reference electrode is not needed for signal correction, some embodiments of the pH sensors described herein may lack a reference electrode altogether. In such embodiments, the second working electrode may be considered as a reference for the first working electrode or vice versa, and a suitable counter electrode may be present to provide a closed electrical circuit.

Moreover, the pH sensors described herein also address the surface fouling issue that may be problematic for conventional electrochemical pH sensors. Namely, the active portions of the first and second working electrodes comprise a relatively thick polymeric layer or spot(s) in which the sensing chemistry takes place throughout the polymeric layer or spot(s), rather than just upon its surface. Passage of electrons through the polymeric layer to the first and second electrodes makes the oxidation-reduction chemistry occur throughout the active portions, rather than just upon the surface. As such, the effects of surface fouling may be limited in the pH sensors disclosed herein. Furthermore, since the oxidation-reduction reaction is not confined to the surface of the active portions in the pH sensors disclosed herein, a variety of mass-limiting or biocompatibilizing membranes may be suitably used in conjunction with the pH sensors, since the interface of the membrane with the active portion does not substantially impact the oxidation-reduction reaction occurring within the interior of the active portion. A variety of proton-permeable membranes may be suitable for use in conjunction with the pH sensors disclosed herein. Suitable proton-permeable membranes may be substantially impermeable to the substances contained within the first and second active portions, thereby promoting retention of those substances in the active portions so that pH-sensing capabilities are retained over extended measurement times.

Finally, the pH sensors of the present disclosure may be desirably operated by conducting a voltammetric sweep of the sensing chemistry (e.g., cyclic voltammetry, differential pulse voltammetry, pulse-wave voltammetry, square-wave voltammetry, and the like) within a given pH measurement range of interest. The measured potential at a given location of the voltammetric sweep using these techniques is substantially invariant of the electrode geometry, such as the thickness and area of the active portion upon each working electrode. As such, manufacturing variances of the active portion are of minimal consequence. In some embodiments, the active portion may comprise sensing spots having an arcuate geometry, such as those described in U.S. Patent Application Publication 2012/0150005 and incorporated herein by reference. Thus, the measured potential is characteristic of the chemistry in each active portion at a given pH. This feature may facilitate calibration of the pH sensors. The measured potential may be an anodic peak potential, a cathodic peak potential, a half-wave potential, or the like, for example, according to one or more embodiments. According to more specific embodiments, the measured potential at each working electrode may comprise the same type of measurement (e.g., the anodic peak potential, the cathodic peak potential, or the half-wave potential of both the first working electrode and the second working electrode) in order to calculate a signal difference.

Accordingly, pH sensors of the present disclosure may comprise a sensor tail comprising a first working electrode, a second working electrode, and at least one other electrode; a first active portion located upon the first working electrode, the first active portion comprising a substance having pH-dependent oxidation-reduction chemistry; and a second active portion located upon the second working electrode, the second active portion comprising a substance having oxidation-reduction chemistry that is substantially invariant with pH. Suitable oxidation-reduction chemistries for the first and second active portions are discussed in further detail hereinbelow.

Various configurations are possible for pH sensors comprising two working electrodes, as discussed hereinafter in reference to the drawings. The at least one other electrode in the pH sensors disclosed herein may comprise a counter electrode or a counter electrode plus a reference electrode, according to some embodiments, and in other embodiments may comprise a counter/reference electrode. Thus, the pH sensors described herein may comprise at least three or at least four electrodes in total, according to various embodiments. Three-electrode configurations are discussed first hereinafter before addressing the four-electrode configurations.

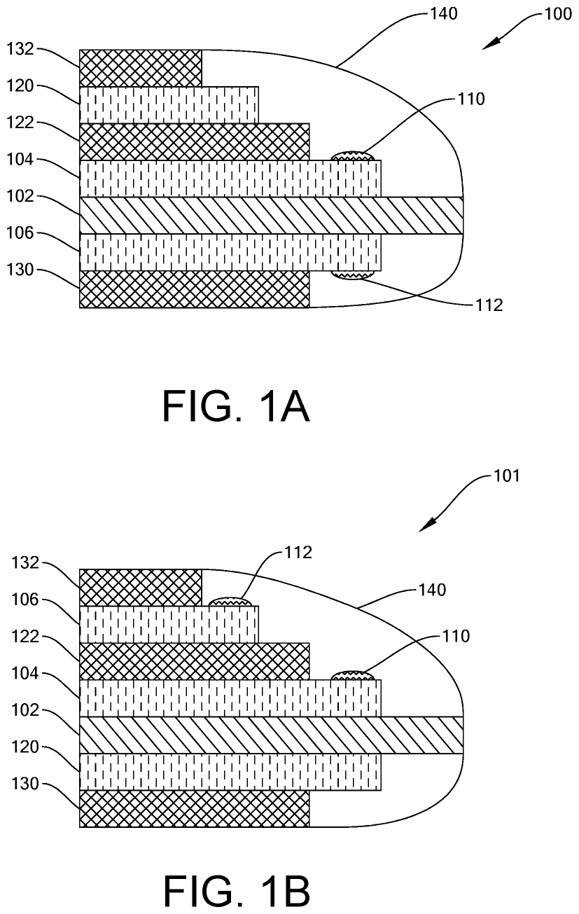

FIGS. 1A and 1B show illustrative configurations of a pH sensor having two working electrodes and a counter/reference electrode, according to the various embodiments described herein. As shown in FIG. 1A, working electrodes 104 and 106 are disposed upon substrate 102 in pH sensor 100. Active portion 110 is disposed upon the surface of working electrode 104, and active portion 112 is disposed upon the surface of working electrode 106. One of active portions 110 and 112 contains a substance having pH-dependent oxidation-reduction chemistry, and the other contains a substance having oxidation-reduction chemistry that is substantially invariant with pH. Counter/reference electrode 120 is electrically isolated from working electrode 104 by dielectric layer 122. Although shown as being positioned upon working electrode 104, it is to be appreciated that counter/reference electrode 120 may be alternately positioned on working electrode 106 in some embodiments. Outer dielectric layers 130 and 132 are positioned upon working electrode 106 and counter/reference electrode 120. It is to be appreciated that the lengths of each layer may vary from that depicted. Active portions 110 and 112 may be exposed so that they can interact with an analyte.

Membrane 140 may overcoat one or both of active portions 110 and 112, according to various embodiments. Membrane 140 may comprise a polymer having biocompatibilizing properties and/or an ability to limit the flux of analyte (i.e., protons) to active portions 110 and 112, according to the disclosure herein. Limiting the analyte flux may be desirable to avoid saturating the sensor. The thickness of membrane 140 may alter the analyte flux, according to various embodiments. The thickness of membrane 140 may vary or remain constant along the length of the sensor. In various embodiments, the thickness of membrane 140 may range between about 1 micron and about 100 microns, or between about microns and about 50 microns, or between about 20 microns and about 90 microns. One or both faces of pH sensor 100, or the whole of pH sensor 100, may be overcoated with membrane 140.

As depicted in FIG. 1A, working electrodes 104 and 106 are positioned on opposite faces of substrate 100. An alternative configuration is shown in FIG. 1B, in which working electrodes 104 and 106 are positioned upon the same face of substrate 102 in pH sensor 101 and are spaced apart by dielectric layer 122. Other alternative configurations also remain within the scope of the present disclosure.

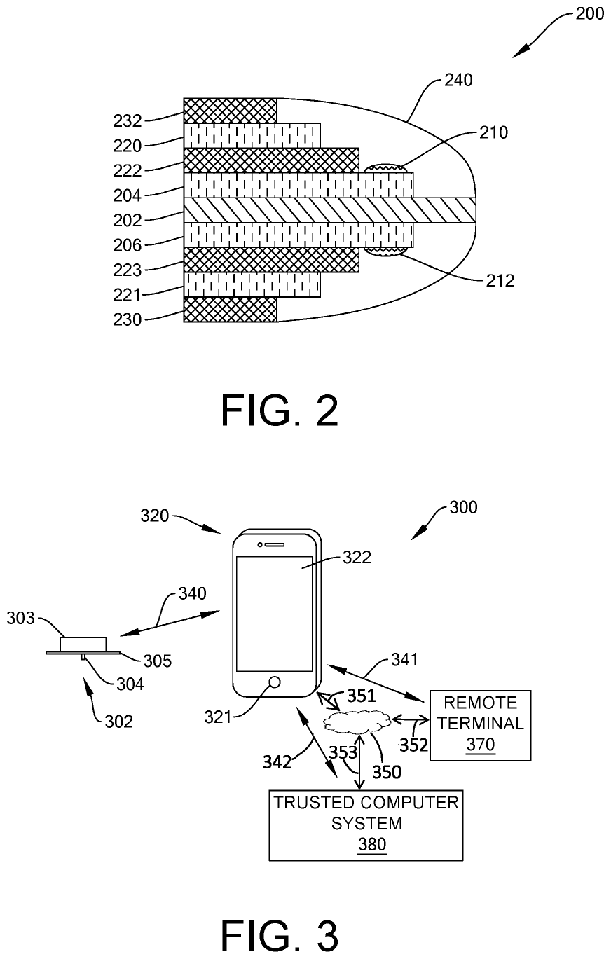

Sensor configurations having both a counter electrode and a reference electrode may be similar in structure to those shown in FIGS. 1A and 1B except for the inclusion of the additional electrode. FIG. 2 shows an illustrative configuration of a pH sensor having two working electrodes, a reference electrode and a counter electrode. As shown, working electrodes 204 and 206 are disposed upon substrate 202 in pH sensor 200. Active portion 210 is disposed upon the surface of working electrode 204, and active portion 212 is disposed upon the surface of working electrode 206. One of active portions 210 and 212 contains a substance having pH-dependent oxidation-reduction chemistry, and the other contains a substance having oxidation-reduction chemistry that is substantially invariant with pH. Counter electrode 220 is electrically isolated from working electrode 204 by dielectric layer 222, and reference electrode 221 is electrically isolated from working electrode 206 by dielectric layer 223. Outer dielectric layers 230 and 232 are positioned upon reference electrode 221 and counter electrode 220, respectively. Membrane 240 may overcoat at least active portions 210 and 212, according to various embodiments. As in FIGS. 1A and 1B, one or both faces of pH sensor 200, or the whole of pH sensor 200, may be overcoated with membrane 240.

The positioning of counter electrode 220 and reference electrode 221 may be reversed from that depicted in FIG. 2. In addition, working electrodes 204 and 206 need not necessarily reside upon opposing faces of substrate 202 in the manner depicted in FIG. 2. As in FIGS. 1A and 1B, one or both faces of pH sensor 200, or the whole of pH sensor 200, may be overcoated with membrane 240.

As mentioned above, one of the active portions may contain a substance having pH-dependent oxidation-reduction chemistry. Suitable substances having pH-dependent oxidation-reduction chemistry include, for example, quinones, redox indicator compounds, or any combination thereof.

Quinones may exhibit a change in oxidation-reduction chemistry and an accompanying difference in observed peak position during a voltammetric sweep as a result of becoming protonated or deprotonated with a change in pH. At low pH values, the phenolic form predominates. As the pH rises and phenol deprotonation begins to occur, oxidation to the quinone form may become favored. Suitable quinones may include, but are not limited to, benzoquinone, naphthoquinone, anthroquinone, 1,10-phenanthrolinequinone, tetrachlorobenzoquinone (chloranil), dichlorodicyanobenzoquinone (DDQ), and the like, functionalized variants thereof, and any combination thereof. In some embodiments, an additional functional group capable of becoming covalently bonded to a working electrode and/or a polymer in an active portion may be present. Suitable quinones having additional functionality may include, but are not limited to, lawsone, alizarin, naphthazirin, and the like, and any combination thereof. Additional functional groups capable of becoming covalently bonded to a working electrode and/or a polymer may be located directly upon the quinone ring or spaced apart therefrom by one or more spacer atoms, such as an alkylene group, an oxyalkylene group, or a carboxylic acid derivative. In some embodiments, suitable quinones may have a structure shown in Formula 1, wherein

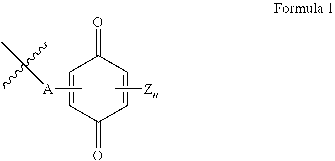

Zₙ represents optional functionality (n=1-4) and A is a spacer group covalently bonded to a polymer comprising the first active portion. In particular embodiments, A may be a spacer group such as, for example, —(CH₂)ₘ—, —C(═O)—NH—, —C(═O)—O—, —O(CH₂)ₘ, or —(CH₂)ₘO—, in which m is a positive integer ranging between 1 and about 20.

Redox indicator compounds include substances that are used in redox titrations based upon their ability to undergo a color change at a specific electrode potential. Specific redox indicator compounds suitable for use in the present disclosure include, but are not limited to, those redox indicator compounds whose oxidation-reduction chemistry is pH dependent. Specific examples include, but are not limited to, indophenol compounds, indigo dyes, phenazines, thiazines, and the like, and any combination thereof. Particular examples of redox indicator compounds having pH-dependent oxidation-reduction chemistry include, but are not limited to, indophenol, sodium 2,6-dibromophenol-indophenol, sodium 2,6-dichlorophenol-indophenol, sodium o-cresol-indophenol, thionine, methylene blue (methylthioninium chloride), toluidine blue, indigotetrasulfonic acid, indigotrisulfonic acid, indigodisulfonic acid (indigo carmine), indigomonosulfonic acid, safranin, phenosafranin, neutral red, and the like, and any combination thereof. Additional functional groups capable of becoming covalently bonded to a working electrode and/or a polymer may be located directly upon the redox indicator compound or spaced apart therefrom by one or more spacer atoms, such as an alkylene chain.

According to some embodiments, a polymer may be present in the first and second active portions. Suitable polymers for inclusion in the first and second active portions may include, but are not limited to, polyvinylpyridines (e.g., poly(4-vinylpyridine)), polyimidazoles (e.g., poly(1-vinylimidazole)), any copolymer thereof, and the like, and any combination thereof. Illustrative copolymers that may be suitable include, but are not limited to, copolymers containing monomer units such as styrene, acrylamide, methacrylamide, acrylonitrile, and the like, and any combination thereof.

In more specific embodiments, the first active portion may comprise a polymer that is covalently bonded to the substance having pH-dependent oxidation-reduction chemistry. In some embodiments, the second active portion may likewise comprise a polymer that is covalently bonded to the substance having oxidation-reduction chemistry that is substantially invariant with pH. The manner in which the substance in the first and second active portions becomes covalently bonded is not considered to be particularly limited and may depend upon the type of polymer or copolymer that is present in the first and second active portions. In some embodiments, the substance may be covalently bonded to the polymer by quaternizing a heterocyclic ring in the polymer (e.g., a pyridine nitrogen atom).

Covalent bonding of the substance having pH-dependent oxidation-reduction chemistry to the polymer comprising the first active portion may take place via a suitable crosslinker. The crosslinker may be introduced through a reaction with a suitable crosslinking agent. Suitable crosslinking agents for reaction with amino or hydroxyl groups in the substance having pH-dependent oxidation-reduction chemistry may include, but are not limited to, polyepoxides such as polyethylene glycol diglycidylether (PEGDGE), cyanuric chloride, N-hydroxysuccinimide, imidoesters, epichlorohydrin, derivatized variants thereof, and the like, and any combination thereof. Suitable crosslinking agents for reaction with carboxylic acid groups in the substance having pH-dependent oxidation-reduction chemistry may include, but are not limited to, carbodiimides.

Suitable substances having oxidation-reduction chemistry that is substantially invariant with pH are not considered to be particularly limited, provided that the substance exhibits a sufficiently limited extent of response variability over a given pH range. According to various embodiments, the response variability may fluctuate by about 100 mV or less, or about 50 mV or less, or about 10 mV or less, or exhibit substantially no fluctuations over a given pH range of interest. In still more specific embodiments, the response variability may fluctuate within the above limits over a pH range of about 1 to about 14, or about 2 to about 12, or about 3 to about 7, or about 7 to about 12, or about 5 to about 8, or about 6.5 to about 8.5, or about 6.5 to about 8.

In still more specific embodiments, the substance having oxidation-reduction chemistry with substantially no pH variability in the second active portion may comprise a transition metal complex, such as the osmium complexes disclosed in, for example, U.S. Pat. Nos. 6,134,461 and 6,605,200, which are incorporated herein by reference in their entirety. The transition metal complex may facilitate conveyance of electrons to the second working electrode during a redox reaction, in which the pH may or may not change. Suitable transition metal complexes may include electroreducible and electrooxidizable ions, complexes or molecules having redox potentials that are a few hundred millivolts above or below the redox potential of the standard calomel electrode (SCE). Other suitable substances for inclusion in the second active portion may comprise complexes of ruthenium, iron (e.g., polyvinylferrocene), or cobalt, for example. Suitable ligands for any of the transition metal complexes may include, but are not limited to, bidentate or higher denticity ligands such as, for example, a bipyridine, biimidazole, pheanthroline, pyridyl(imidazole), and the like, and any combination thereof. Other suitable bidentate ligands may include, but are not limited to, amino acids, oxalic acid, acetylacetone, diaminoalkanes, o-diaminoarenes, and the like, and any combination thereof. Any combination of monodentate, bidentate, tridentate, tetradentate, or higher denticity ligands may be present in the metal complex to achieve a full coordination sphere. One or more of the ligands in the metal complex may also be covalently bonded to the polymer in the second active portion.

In other embodiments, the substance having substantially no variability in oxidation-reduction chemistry with respect to pH in the second active portion may be a pH-independent redox indicator. Suitable pH-independent redox indicators may include, for example, Ru 2,2′-bipyridine, Fe 2,2′-bipyridine, nitrophenanthroline, N-phenylanthranilic acid, 1,10-phenanthroline iron sulfate complex, N-ethoxychysoidine, Fe 5,6-dimethylphenanthroline, o-dianisidine, sodium diphenylamine sulfonate, diphenylbenzidine, diphenylamine, and viologen. Other substances having oxidation-reduction chemistry with substantially no pH variability may include any substance that does not undergo protonation or deprotonation over the pH range of interest, provided that the oxidation-reduction reaction is reversible.

Although the substances in the first and second active portions may be covalently bonded to a polymer within each active portion, other association means to the polymer may be suitable as well. In some embodiments, the substances may be ionically or coordinatively associated with the polymer. For example, a charged polymer may be ionically associated with an oppositely charged substance. In still other embodiments, the substance may be physically entrained within the polymer without being bonded thereto.

According to various embodiments of the present disclosure, each working electrode is configured to produce a signal, such that a difference between the signals may be correlated to a pH value. Before further describing how the signal difference is determined and correlated to a pH value, a brief description of how the pH sensors may communicate the signals to a user or a processor for further analysis is provided.

According to various embodiments, pH sensors of the present disclosure may be adapted for on-body wear, such that the sensor tail is configured for insertion in a tissue, particularly dermally, subcutaneously, or interstitially below the skin. FIG. 3 shows a diagram of an illustrative sensing system adapted for on-body wear and capable of measuring pH based upon receipt of signals from first and second working electrodes, according to the present disclosure. It is to be appreciated, however, that pH sensors having architectures, configurations, and/or components different than or in addition to those described expressly hereinafter may also be used suitably in some embodiments of the present disclosure.

As shown in FIG. 3, sensing system 300 includes sensor control device 302 and reader device 320 that are configured to communicate with one another over a local or remote communication path or link, which may be wired or wireless, uni- or bi-directional, and encrypted or non-encrypted. Reader device 320 may constitute an output medium for viewing pH and alerts or notifications determined by sensor 304 or a processor associated therewith, as well as allowing for one or more user inputs, according to some embodiments. Reader device 320 may be a multi-purpose smartphone or a dedicated electronic reader instrument. While only one reader device 320 is shown, more than one reader device 320 may be present in certain instances. Multiple reader devices 320 may be in communication with one another (e.g., to share and synchronize data). Reader device 320 may also be in communication with remote terminal 370 and/or trusted computer system 380 via communication path(s)/link(s) 341 and/or 342, respectively, which also may be wired or wireless, uni- or bi-directional, and encrypted or non-encrypted. Reader device 320 may also or alternately be in communication with network 350 (e.g., a mobile telephone network, the internet, or a cloud server) via communication path/link 351. Network 350 may be further communicatively coupled to remote terminal 370 via communication path/link 352 and/or trusted computer system 380 via communication path/link 353. Remote terminal 370 and/or trusted computer system 380, in turn, may communicate with network 350, in some embodiments. Alternately, sensor 302 may communicate directly with remote terminal 370 and/or trusted computer system 380 without an intervening reader device 320 being present. For example, sensor 302 may communicate with remote terminal 370 and/or trusted computer system 380 through a direct communication link to network 350, according to some embodiments, as described in U.S. Patent Application Publication 2011/0213225 and incorporated herein by reference in its entirety. Any suitable electronic communication protocol may be used for each of the communication paths or links, such as near field communication (NFC), radio frequency identification (RFID), BLUETOOTH® or BLUETOOTH® Low Energy protocols, WiFi, or the like. Remote terminal 370 and/or trusted computer system 380 may be accessible, according to some embodiments, by individuals other than a primary user. Reader device 320 may comprise display 322 and optional input component 321. Display 322 may comprise a touch-screen interface, according to some embodiments.

Sensor control device 302 includes sensor housing 303, which may house circuitry and a power source for operating sensor 304. Optionally, the power source and/or active circuitry may be omitted. A processor (not shown) may be communicatively coupled to sensor 304, with the processor being physically located within sensor housing 303 or reader device 320. Sensor 304 protrudes from the underside of sensor housing 303 and extends through adhesive layer 305, which is adapted for adhering sensor housing 303 to a tissue surface, such as skin, according to some embodiments.

Sensor 304 is adapted to be at least partially inserted into a tissue of interest, such as below the skin. Sensor 304 may comprise a sensor tail of sufficient length for insertion to a desired depth below the skin. The sensor tail may comprise a sensing region having two working electrodes according to the disclosure herein, according to one or more embodiments. In various embodiments of the present disclosure, pH may be monitored in any biological fluid of interest such as dermal fluid, interstitial fluid, plasma, blood, lymph, synovial fluid, cerebrospinal fluid, saliva, bronchoalveolar lavage, amniotic fluid, or the like.

An introducer may be present transiently to promote introduction of sensor 304 into a tissue. In illustrative embodiments, the introducer may comprise a needle. It is to be recognized that other types of introducers, such as sheaths or blades, may be present in alternative embodiments. More specifically, the needle or similar introducer may transiently reside in proximity to sensor 304 prior to insertion and then be withdrawn afterward. While present, the needle or other introducer may facilitate insertion of sensor 304 into a tissue by opening an access pathway for sensor 304 to follow. For example, the needle may facilitate penetration of the epidermis as an access pathway to the dermis to allow implantation of sensor 304 to take place, according to one or more embodiments. After opening the access pathway, the needle or other introducer may be withdrawn so that it does not represent a sharps hazard. In illustrative embodiments, the needle may be solid or hollow, beveled or non-beveled, and/or circular or non-circular in cross-section. In more particular embodiments, the needle may be comparable in cross-sectional diameter and/or tip design to an acupuncture needle, which may have a cross-sectional diameter of about 250 microns. It is to be recognized, however, that suitable needles may have a larger or smaller cross-sectional diameter if needed for particular applications.

In some embodiments, a tip of the needle (while present) may be angled over the terminus of sensor 304, such that the needle penetrates a tissue first and opens an access pathway for sensor 304. In other illustrative embodiments, sensor 304 may reside within a lumen or groove of the needle, with the needle similarly opening an access pathway for sensor 304. In either case, the needle is subsequently withdrawn after facilitating insertion.

Accordingly, in the pH sensors of the present disclosure, the first working electrode may be configured to produce a first signal and the second working electrode may be configured to produce a second signal, such that a difference between the first signal and the second signal may be correlated with pH. That is, according to various embodiments of the present disclosure, by subtracting the second signal from the first signal, the difference in signal magnitude may be correlated to pH. The signal difference may be calculated manually or automatically with a suitable processor. Likewise, once calculated, the signal difference may be correlated either manually or automatically with a processor.

In more specific embodiments, the pH sensors of the present disclosure may comprise a processor in signal communication with the first and second working electrodes. The processor may be configured to receive a first signal from the first working electrode and a second signal from the second working electrode. The processor may be further configured to calculate the difference between the first signal and the second signal, and to correlate the difference between the signals to pH.

In some embodiments, the processor may be configured to access a lookup table comprising a plurality of pH values and corresponding differences between the first signal and the second signal in order to calculate pH. The lookup table may be populated before measuring an unknown sample by assaying multiple samples with known pH, measuring the first and second signals, and determining the difference between the two. The processor may, for example, determine which difference value in the lookup table is closest to that measured for the known sample and then report the pH accordingly. In other embodiments, the processor may interpolate between the difference values in the lookup table to determine a measured pH value. Interpolation may assume a linear variance in pH between the reported difference values.

In other embodiments, the processor may be configured to access a calibration curve of pH values versus corresponding differences between the first signal and the second signal in order to calculate pH. Like a lookup table, the calibration curve may be determined before measuring an unknown sample by assaying multiple samples with known pH, measuring the first and second signals, determining the difference between the two, and curve fitting the pH and difference values to determine a calibration function. Factory calibration at the lot level may be possible by determining the signal difference as a function of pH at the factory and assigning a lookup table or calibration curve to the pH sensor. Since reference electrode correction of each signal is not necessary, the signal difference over a given pH range should be invariant from sensor to sensor for a given selection of substances in the first active portion and the second active portion.

Accordingly, pH measurement methods of the present disclosure may comprise: exposing a pH sensor to a fluid having a pH value, the pH sensor comprising a first working electrode, a second working electrode, and at least one other electrode, as described above; measuring a first signal associated with the first working electrode; measuring a second signal associated with the second working electrode; calculating a difference between the first signal and the second signal; and correlating the difference between the first signal and the second signal to the pH value. In more specific embodiments, the fluid may be a biological fluid and the pH sensor may be exposed to the biological fluid in vivo.

According to some embodiments, the first signal and the second signal may each comprise a voltammetric peak potential. The voltammetric peak potential may be determined by cyclic voltammetry, differential pulse voltammetry, pulse-wave voltammetry, square-wave voltammetry, or the like. Depending upon the type of voltammetric sweep performed, an appropriate location upon the observed curve to determine the voltammetric peak potential for each substance can be determined. One having ordinary skill in the art will be able to make this determination based upon the type of voltammetric sweep being performed.

The first signal and the second signal may be measured at the same time or at different times, according to various embodiments. Measurement at different times may comprise, for example, performing a voltammetric sweep of each working electrode separately, without applying a potential to the other working electrode. Measurement in this manner may utilize a single channel, according to some embodiments. In other embodiments, the first signal and the second signal may be measured at the same time by monitoring each working electrode simultaneously via a first channel and a second channel.

In further embodiments, methods of the present disclosure may comprise accessing a lookup table comprising a plurality of pH values and corresponding differences between the first signal and the second signal in order to calculate pH. In other further embodiments, methods of the present disclosure may comprise accessing a calibration curve of pH values versus corresponding differences between the first signal and the second signal in order to calculate pH. In either configuration, a processor may be configured to receive the first signal and the second signal, to calculate the difference between the first signal and the second signal, and to access the lookup table or calibration curve. Accessing the lookup table or calibration curve may comprise performing an electronic query, according to various embodiments.

Embodiments disclosed herein include:

A. Sensors for measuring pH. The pH sensors comprise: a sensor tail comprising a first working electrode, a second working electrode, and at least one other electrode; a first active portion located upon the first working electrode, the first active portion comprising a substance having pH-dependent oxidation-reduction chemistry; and a second active portion located upon the second working electrode, the second active portion comprising a substance having oxidation-reduction chemistry that is substantially invariant with pH.

B. Methods for measuring pH. The methods comprise: exposing a pH sensor to a fluid having a pH value, the pH sensor comprising: a sensor tail comprising a first working electrode, a second working electrode, and at least one other electrode; a first active portion located upon the first working electrode, the first active portion comprising a substance having pH-dependent oxidation-reduction chemistry; and a second active portion located upon the second working electrode, the second active portion comprising a substance having oxidation-reduction chemistry that is substantially invariant with pH; measuring a first signal associated with the first working electrode; measuring a second signal associated with the second working electrode; calculating a difference between the first signal and the second signal; and correlating the difference between the first signal and the second signal to the pH value.

Each of embodiments A and B may have one or more or all of the following additional elements in any combination:

Element 1: wherein the sensor tail is configured for insertion in a tissue.

Element 2: wherein the substance having pH-dependent oxidation-reduction chemistry comprises a quinone, a redox indicator compound, or any combination thereof.

Element 3: wherein the substance having pH-dependent oxidation-reduction chemistry comprises a redox indicator compound comprising a thiazine.

Element 4: wherein the at least one other electrode comprises a counter electrode and a reference electrode.

Element 5: wherein the pH sensor further comprises: a dielectric layer interposed between the at least one other electrode and at least one of the first working electrode and the second working electrode.

Element 6: wherein a first dielectric layer is interposed between the first working electrode and the counter electrode or the reference electrode and a second dielectric layer is interposed between the second working electrode and the counter electrode or the reference electrode.

Element 7: wherein the at least one other electrode comprises a counter/reference electrode.

Element 8: wherein the pH sensor further comprises: a dielectric layer interposed between the counter/reference electrode and at least one of the first working electrode and the second working electrode.

Element 9: wherein the first working electrode is configured to produce a first signal and the second working electrode is configured to produce a second signal, and a difference between the first signal and the second signal correlates to pH.

Element 10: wherein the pH sensor further comprises: a processor configured to receive the first signal from the first working electrode and the second signal from the second working electrode; wherein the processor is further configured to calculate the difference between the first signal and the second signal, and to correlate the difference to pH.

Element 11: wherein the processor is configured to access a lookup table comprising a plurality of pH values and corresponding differences between the first signal and the second signal in order to calculate pH.

Element 12: wherein the processor is configured to access a calibration curve of pH values versus corresponding differences between the first signal and the second signal in order to calculate pH.

Element 13: wherein the substance having pH-dependent oxidation-reduction chemistry and the substance having oxidation-reduction chemistry that is substantially invariant with pH are both covalently bound to a polymer in the first active portion and the second active portion, respectively.

Element 14: wherein the fluid is a biological fluid and the pH sensor is exposed to the biological fluid in vivo.

Element 15: wherein the method further comprises: accessing a lookup table comprising a plurality of pH values and corresponding differences between the first signal and the second signal in order to calculate pH.

Element 16: wherein a processor is configured to receive the first signal and the second signal, to calculate the difference between the first signal and the second signal, and to access the lookup table.

Element 17: wherein the method further comprises: accessing a calibration curve of pH value versus corresponding differences between the first signal and the second signal in order to calculate pH.

Element 18: wherein a processor is configured to receive the first signal and the second signal, to calculate the difference between the first signal and the second signal, and to access the calibration curve.

Element 19: wherein the first signal comprises a voltammetric peak potential of the substance having pH-dependent oxidation-reduction chemistry and the second signal comprises a voltammetric peak potential of the substance having oxidation-reduction chemistry that is substantially invariant with pH.

Element 20: wherein the first signal and the second signal are measured at different times.

Element 21: wherein the first signal and the second signal are measured simultaneously via a first channel and a second channel.

By way of non-limiting example, exemplary combinations applicable to A and B include:

The pH sensor of A in combination with elements 1 and 2; 1 and 3; 1 and 4; 1, 4 and 5; 1, 4 and 6; 1 and 7; 1, 7 and 8; 1 and 9; 1 and 10; 1, 10 and 11; 1, 10 and 12; 1 and 13; 2 and 4; 2, 4 and 5; 2, 4 and 6; 2 and 7; 2, 7 and 8; 2 and 9; 2 and 10; 2, 10 and 11; 2, 10 and 12; 3 and 4; 3, 4 and 5; 3, 4 and 6; 3 and 7; 3, 7 and 8; 3 and 9; 3 and 10; 3, 10 and 11; 3, 10 and 12; 4 and 10; 4, 10 and 11; 4, 10 and 12; 7 and 10; 7, 10 and 11; 7, 10 and 12; 2 and 9; 3 and 9; 4 and 9; 7 and 9; 10 and 11; 10 and 12; 10 and 13; 10, 11 and 13; and 10, 12 and 13. The method of B in combination with elements 2 and 13; 2 and 14; 2 and 15; 2, 15 and 16; 2 and 17; 2, 17 and 18; 2 and 19; 2 and 20; 2 and 21; 3 and 13; 3 and 14; 3 and 15; 3, 15 and 16; 3 and 17; 3, 17 and 18; 3 and 19; 3 and 20; 3 and 21; 13 and 14; 13 and 15; 13, 15 and 16; 13 and 17; 13, 17 and 18; 13 and 19; 13 and 20; 13 and 21; 14 and 15; 14, 15 and 16; 14 and 17; 14, 17 and 18; 14 and 19; 14 and 20; 14 and 21; 15 and 16; 15 and 19; 15, 16 and 19; 15 and 20; 15, 16 and 20; 15 and 21; 15, 16 and 21; 17 and 19; 17, 18 and 19; 17 and 20; 17, 18 and 20; 17 and 21; 17, 18 and 21; 19 and 20; and 19 and 21.

To facilitate a better understanding of the embodiments described herein, the following examples of various representative embodiments are given. In no way should the following examples be read to limit, or to define, the scope of the invention.

### Examples

Working Electrode #1:

A first carbon working electrode was coated with a polymerized layer of toluidine blue (TOB). Before coating, the bare working electrode was pre-conditioned in a solution comprising 100 mM citric acid/200 mM phosphate/100 mM KCl (pH=4) by cyclically sweeping the potential from −0.8 V to 1.2 Vat a sweep rate of 50 mV/s for 5 cycles. Electrodeposition of TOB was carried out by adding 5 mM TOB to the above solution and cycling the potential from −0.8 V to 1.2 V at a sweep rate of 50 mV/s for 60 cycles. The sensor was then rinsed with distilled water and air-dried.

Working Electrode #2:

A second carbon working electrode was coated with a polymer having an osmium complex covalently attached thereto. The structure of the polymer, which is described in further detail in U.S. Pat. No. 6,605,200 and incorporated herein in its entirety, is shown below in Formula 2.

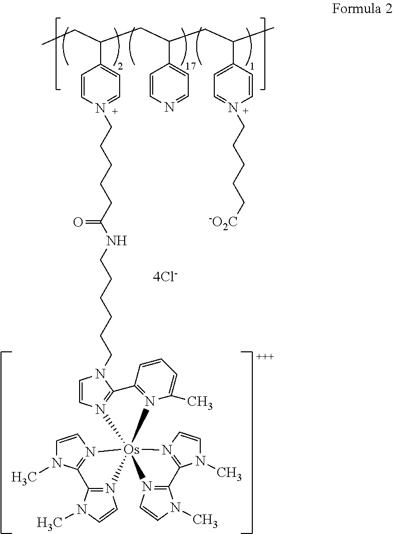

A solution having 45 mg/mL of the above polymer and 15 mg/mL of PEG400 was freshly prepared in 10 mM HEPES buffer (pH=8). Three 20 nL aliquots of the solution were applied to the electrode surface to produce 3 sensing (active) layer spots, each having an approximate area of 0.1 mm². The working electrode was then cured overnight at 65% relative humidity at 25° C.

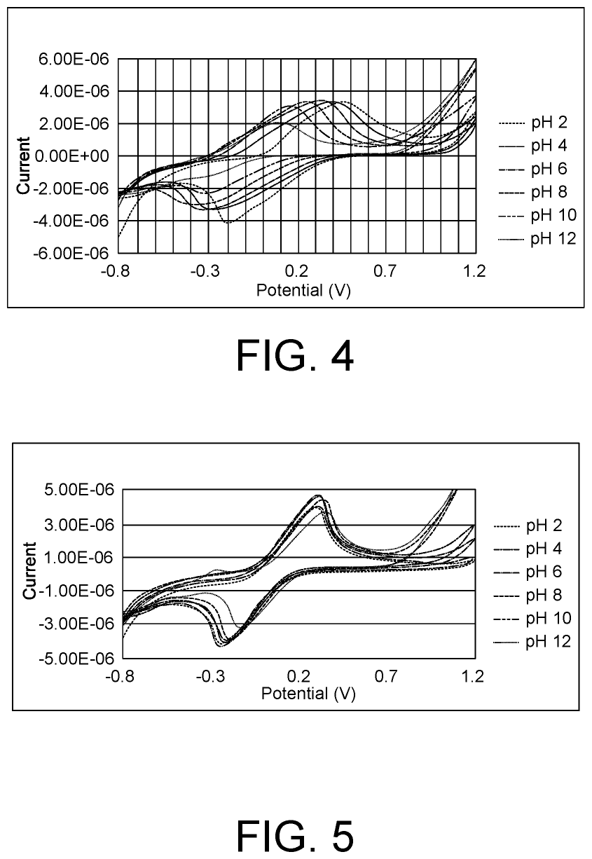

Cyclic voltammetry was performed separately on each working electrode over a potential range of −0.8 V to 1.2 V at a scan rate of 50 mV/s in a series of pH buffers having a pH ranging between 2 and 12. A carbon counter electrode and a Ag/AgCl reference electrode were used in making all measurements. FIG. 4 shows a plot of aggregate cyclic voltammograms at various pH values for the first working electrode (containing TOB). FIG. 5 shows a corresponding plot of aggregate cyclic voltammograms at various pH values for the second working electrode (containing the polymer-bound osmium complex). Table 1 below summarizes the observed anodic peak potentials.

TABLE 1

| | pH = 2 | pH = 4 | pH = 6 | pH = 8 | pH = 10 | pH = 12 |
|---|---|---|---|---|---|---|
| Electrode 1 | 0.459 | 0.401 | 0.337 | 0.252 | 0.159 | 0.101 |
| Anodic Peak | | | | | | |
| Potential (V) | | | | | | |
| Electrode 2 | 0.301 | 0.310 | 0.312 | 0.343 | 0.317 | 0.353 |
| Anodic Peak | | | | | | |
| Potential (V) | | | | | | |
| Difference (V) | 0.158 | 0.091 | 0.025 | −0.091 | −0.158 | −0.252 |

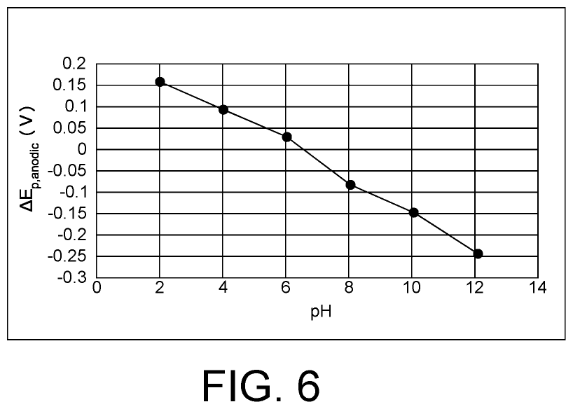

The difference values in the bottom row of Table 1 and the corresponding pH values in the top row may constitute a lookup table. Alternately, a calibration curve may be constructed by plotting the values. FIG. 6 shows a calibration curve corresponding to the pH versus voltage difference data shown in Table 1.

Unless otherwise indicated, all numbers expressing quantities and the like in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

One or more illustrative embodiments incorporating various features are presented herein. Not all features of a physical implementation are described or shown in this application for the sake of clarity. It is understood that in the development of a physical embodiment incorporating the embodiments of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

While various systems, tools and methods are described herein in terms of “comprising” various components or steps, the systems, tools and methods can also “consist essentially of” or “consist of” the various components and steps.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Therefore, the disclosed systems, tools and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems, tools and methods illustratively disclosed herein may suitably be practiced in the absence of any element that is not specifically disclosed herein and/or any optional element disclosed herein. While systems, tools and methods are described in terms of “comprising,” “containing,” or “including” various components or steps, the systems, tools and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

What is claimed is the following:

1. A pH sensor comprising:
a sensor tail comprising a first working electrode, a second working electrode, and at least one other electrode;
a first active portion located upon the first working electrode, the first active portion comprising a substance having pH-dependent oxidation-reduction chemistry; and
a second active portion located upon the second working electrode, the second active portion comprising a substance having oxidation-reduction chemistry that is substantially invariant with pH.

2. The pH sensor of claim 1, wherein the sensor tail is configured for insertion in a tissue.

3. The pH sensor of claim 1, wherein the substance having pH-dependent oxidation-reduction chemistry comprises a quinone, a redox indicator compound, or any combination thereof.

4. The pH sensor of claim 3, wherein the substance having pH-dependent oxidation-reduction chemistry comprises a redox indicator compound comprising a thiazine.

5. The pH sensor of claim 1, wherein the at least one other electrode comprises a counter electrode and a reference electrode and a dielectric layer interposed between the at least one other electrode and at least one of the first working electrode and the second working electrode.

6. The pH sensor of claim 5, wherein a first dielectric layer is interposed between the first working electrode and the counter electrode or the reference electrode and a second dielectric layer is interposed between the second working electrode and the counter electrode or the reference electrode.

7. The pH sensor of claim 1, wherein the at least one other electrode comprises a counter/reference electrode and a dielectric layer interposed between the counter/reference electrode and at least one of the first working electrode and the second working electrode.

8. The pH sensor of claim 1, wherein the first working electrode is configured to produce a first signal and the second working electrode is configured to produce a second signal, and a difference between the first signal and the second signal correlates to pH.

9. The pH sensor of claim 8, further comprising:
a processor configured to receive the first signal from the first working electrode and the second signal from the second working electrode;
wherein the processor is further configured to calculate the difference between the first signal and the second signal, and to correlate the difference to pH.

10. The pH sensor of claim 9, wherein the processor is configured to (1) access a lookup table comprising a plurality of pH values and corresponding differences between the first signal and the second signal in order to calculate pH or (2) access a calibration curve of pH values versus corresponding differences between the first signal and the second signal in order to calculate pH.

11. The pH sensor of claim 1, wherein the substance having pH-dependent oxidation-reduction chemistry and the substance having oxidation-reduction chemistry that is substantially invariant with pH are both covalently bound to a polymer in the first active portion and the second active portion, respectively.

12. A method comprising:
exposing a pH sensor to a fluid having a pH value, the pH sensor comprising:
a sensor tail comprising a first working electrode, a second working electrode, and at least one other electrode;
a first active portion located upon the first working electrode, the first active portion comprising a substance having pH-dependent oxidation-reduction chemistry; and
a second active portion located upon the second working electrode, the second active portion comprising a substance having oxidation-reduction chemistry that is substantially invariant with pH;
measuring a first signal associated with the first working electrode;
measuring a second signal associated with the second working electrode;
calculating a difference between the first signal and the second signal; and
correlating the difference between the first signal and the second signal to the pH value.

13. The method of claim 12, wherein the fluid is a biological fluid and the pH sensor is exposed to the biological fluid in vivo.

14. The method of claim 12, wherein the substance having pH-dependent oxidation-reduction chemistry comprises a quinone, a redox indicator compound, or any combination thereof.

15. The method of claim 12, further comprising:
accessing a lookup table comprising a plurality of pH values and corresponding differences between the first signal and the second signal in order to calculate pH.

16. The method of claim 15, wherein a processor is configured to receive the first signal and the second signal, to calculate the difference between the first signal and the second signal, and to access the lookup table.

17. The method of claim 12, further comprising:
accessing a calibration curve of pH value versus corresponding differences between the first signal and the second signal in order to calculate pH.

18. The pH sensor of claim 17, wherein a processor is configured to receive the first signal and the second signal, to calculate the difference between the first signal and the second signal, and to access the calibration curve.

19. The method of claim 12, wherein the first signal comprises a voltammetric peak potential of the substance having pH-dependent oxidation-reduction chemistry and the second signal comprises a voltammetric peak potential of the substance having oxidation-reduction chemistry that is substantially invariant with pH.

20. The method of claim 12, wherein the first signal and the second signal are measured at different times or are measured simultaneously via a first channel and a second channel.
